(19) United States
(12) Patent Application Publication
(10) Pub. No.: US 2024/0424152 A1
(43) Pub. Date: Dec. 26, 2024

(54) AGENTS, COMPOSITIONS, AND METHODS FOR CANCER DETECTION

(71) Applicant: The Johns Hopkins University, Baltimore, MD (US)

(72) Inventor: Babak Behnam Azad, Towson, MD (US)

(21) Appl. No.: 18/294,646

(22) PCT Filed: Aug. 3, 2022
(86) PCT No.: PCT/US2022/074463

§ 371 Date: Feb. 2, 2024

Related U.S. Application Data
(60) Provisional application No. 63/228,841, filed on Aug. 3, 2021.

(51) Int. Cl. A61K 51/08; A61P 35/00

(52) U.S. Cl. CPC A61K 51/088; A61P 35/00; A61K 2123/00

(57) ABSTRACT

The present disclosure provides agents for detecting cancer based on peptides derived from thymosin beta-10 (Tβ-10) and compositions and methods of use thereof. Particularly, the disclosure provides agents comprising a peptide derived from thymosin beta-10 (Tβ-10) covalently attached to an imaging agent, wherein the peptide comprises less than 20 amino acids and methods of detecting, diagnosing, or imaging cancer.

### CROSS-REFERENCE TO RELATED APPLICATIONS

This application claims the benefit of U.S. Provisional Application No. 63/228,841, filed Aug. 3, 2021, the contents of which are herein incorporated by reference in their entirety.

### FIELD

The present disclosure provides agents for detecting cancer based on peptides derived from thymosin beta-10 (Tβ-10) and compositions, conjugates, and methods of use thereof.

### SEQUENCE LISTING STATEMENT

The contents of the electronic sequence listing titled (JHU-39633.601.xml; Size: 4,287 bytes; and Date of Creation: Aug. 3, 2022) is herein incorporated by reference in its entirety.

### BACKGROUND

Despite ongoing advances in targeted therapies, women with metastatic breast cancer still have a limited survival time of 18-24 months, with a 5-year survival rate of only 20% post initial diagnosis of distant metastases. A crucial factor in patient outcome is the time of diagnosis as early detection is associated with decreased mortality. A late-stage diagnosis, on the other hand, is accompanied by longer waiting times prior to initiation of therapy while the disease continues to progress to metastasis.

Mammography remains the current standard for detection of primary breast tumors. Although this technique is still the front-line method for breast cancer screening, it exhibits significant limitations with regards to sensitivity, particularly in women with greater mammographic breast densities and alterations in breast density over time, owing to stromal and epithelial tissues decreasing with age and menopause, rapid tumor growth rates and hormone replacement therapies, which further limit mammographic sensitivity.

Position emission tomography (PET) with [¹⁸F] fluorodeoxyglucose ([¹⁸F] FDG), an indicator of glycolic metabolism, is also utilized for breast cancer detection. However, significant accumulation of FDG in non-neoplastic conditions, such as infections and inflammations, as well as its non-specific uptake in brown adipose tissues, commonly observed in patients with breast cancer. limit the utility of FDG-PET for breast cancer detection.

### SUMMARY

Disclosed herein are agents for detecting cancer, comprising a peptide derived from thymosin beta-10 (Tβ-10) covalently attached to an imaging agent. In some embodiments, the peptide comprises less than twenty amino acids. In some embodiments, the peptide comprises the amino acid sequence ADKPDMGEIASFDK (SEQ ID NO: 1).

In some embodiments, the imaging agent is selected from the group consisting of a radionuclide, a chelator, a radiopaque agent, a radiolucent agent, a contrast agent, a metal, quantum dot, or a combination thereof.

In certain embodiments, the imaging agent comprises a chelator. In select embodiments, the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

In certain embodiments, the imaging agent comprises a radionuclide. In select embodiments, the radionuclide is a gamma or positron emitting radionuclide. In some embodiments, the radionuclide is ¹²³I, ^{99m}Tc, ¹⁸F or ¹²⁴I. In exemplary embodiments, the radionuclide is ISF.

Also disclosed herein is a composition comprising one or more agent for detecting cancer, as described herein.

Further disclosed are conjugates comprising an agent of claim, wherein the imaging agent is a chelator, and a radionuclide bound to the chelator.

In some embodiments, the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

In select embodiments, the radionuclide is a gamma or positron emitting radionuclide. In some embodiments, the radionuclide is ¹²³I, ^{99m}Tc, ¹⁸F or ¹²⁴I. In exemplary embodiments, the radionuclide is ¹⁸F.

Further disclosed are methods of detecting or diagnosing cancer comprising contacting one or more cells, organs, or tissues with an effective amount of an agent, a composition, or a conjugate, as described herein. In some embodiments, the methods further comprise identifying the agent. In some embodiments, the contacting comprises administering the agent, composition, or conjugate to a subject.

In some embodiments, the identifying comprises: exposing the subject or a region of the subject to an image scanner; obtaining an image of the subject or the region of the subject; and recognizing the agent.

In some embodiments, the subject has or is suspected of having cancer. In some embodiments, the cancer comprises a solid tumor. In some embodiments, the cancer comprises breast cancer.

Additionally disclosed are kits comprising an agent, as described herein, wherein the imaging agent is a chelator and a radionuclide.

In some embodiments, the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

In select embodiments, the radionuclide is a gamma or positron emitting radionuclide. In some embodiments, the radionuclide is ¹²³I, ^{99m}Tc, ¹⁸F or ¹²⁴I. In exemplary embodiments, the radionuclide is ¹⁸F.

Other aspects and embodiments of the disclosure will be apparent in light of the following detailed description and accompanying figures.

### BRIEF DESCRIPTION OF THE DRAWINGS

FIG. 1 is a graph of the cellular uptake of exemplary peptides (Pep61 and Pep62) in the human triple negative breast cancer cell line MDA-MB-231, along with blocking using an excess of unlabeled peptide to show specific binding.

FIG. 2 is non-invasive in vivo PET images of radiolabeled Pep61 and Pep62 thirty minutes after intravenous administration in mice xenografted with human triple negative breast tumors derived from MDA-MB-231 cells. Accumulation and visualization of both peptides in breast tumors, as indicated by arrows, demonstrated the applications of these compounds for targeted breast cancer imaging. Results also show higher and more uniform uptake of Pep62 compared to Pep61.

FIGS. 3A and 3B are graphs of ex vivo biodistribution of [¹⁸F] Pep61 (FIG. 3A) and [¹⁸F] Pep62 (FIG. 3B) in a mouse model of human triple negative breast cancer (MDA-MB-231). Uptake was seen in tumors as well as non-specific retention in clearance organs. Higher overall tumor uptake, as well as tumor-to-blood and tumor-to-muscle ratios was observed for [¹⁸F] Pep62 compared to [¹⁸F] Pep61.

### DETAILED DESCRIPTION

The present disclosure provides clinically translatable peptide imaging agents based on or derived from the sequence of thymosin beta-10 (Tβ-10). The imaging agents were evaluated in a mouse model of human triple negative breast cancer using positron emission tomography (PET). Both agents were preferentially taken up by breast tumors and showed a promising biodistribution profile, irrespective of the presence of hormone receptors (ER, PR, and HER2). These imaging agents may facilitate early detection of breast cancer in a broader range of patients.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The recitations “sequence identity,” “percent identity,” “percent homology,” “percent similarity,” or, for example, comprising a “sequence 50% identical to” or “sequence with at least 50% similarity to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (e.g., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4:11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine (Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry. 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D) Phe, D-Phe, or ^{D}F for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

The term “pharmaceutically acceptable salt” in the context of the present invention (pharmaceutically acceptable salt of a peptide described herein) refers to a salt which is not harmful to a patient or subject to which the salt in question is administered. It may suitably be a salt chosen, e.g., among acid addition salts and basic salts. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the peptides may also be quaternized with alkyl chlorides, bromides, and iodides such as methyl, ethyl, propyl, isopropyl, butyl, lauryl, myristyl, stearyl and the like. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66:2 (1977).

As used herein, the terms “providing,” “administering,” and “introducing,” are used interchangeably herein and refer to the placement of the peptides or compositions of the disclosure into a subject by a method or route which results in at least partial localization to a desired site. The peptides or compositions can be administered by any appropriate route which results in delivery to a desired location in the subject.

The term “solvate” in the context of the present invention refers to a complex of defined stoichiometry formed between a solute (the peptide or pharmaceutically acceptable salt thereof described) and a solvent. The solvent in this connection may, for example, be water, ethanol, or another pharmaceutically acceptable, typically small-molecular organic species, such as, but not limited to, acetic acid or lactic acid. When the solvent in question is water, such a solvate is normally referred to as a hydrate.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Cancer Detecting Agent

The present disclosure provides an agent for detecting cancer, comprising a peptide derived from thymosin beta-10 (Tβ-10), or a pharmaceutically acceptable salt or solvate thereof, covalently attached to an imaging agent. The invention is not limited by which species the thymosin beta-10 (Tβ-10) peptide is derived. Preferably, the peptide is derived from human thymosin beta-10 (Tβ-10), e.g., NCBI Reference Sequence: NP_066926.1.

In some embodiments, the peptide comprises less than twenty amino acids. In some embodiments, the peptide comprises 10-20 amino acids. In some embodiments, the peptide comprises 10-20 amino acids. In some embodiments, the peptide comprises 10, 11, 12, 13, 14, 15, 16, 17, 18, or 19 amino acids.

In some embodiments, the peptide comprises the amino acid sequence ADKPDMGEIASFDK (SEQ ID NO: 1).

In some embodiments, the peptide comprises an amino acid sequence with 1, 2, 3, 4, 5, 6, 7, or 8 substitutions to SEQ ID NO:1. The substitutions may be conservative substitutions (e.g., one or more amino acids are replaced by another, biologically similar residue defined by polarity, charge, acidity, hydrophobicity, or chemical structure (e.g., aromaticity)), radical substitutions (e.g., one or more amino acids are replaced by residue with different physiochemical properties defined by polarity, charge, acidity, hydrophobicity, or chemical structure (e.g., aromaticity)), or a combination thereof.

The peptide may be further modified by the addition of: affinity tags (e.g., His tag, biotin); PEG moieties; carbohydrates (e.g., glycosylation, hesylation); and organic molecules (e.g., alkylation, acetylation, acylation).

The present disclosure also provides for use of nonpeptide compounds that mimic peptide sequences (“mimetics”) in the agent, synthesis of which are known in the art. Peptide mimetics that are structurally related to therapeutically useful peptides may be used to produce an equivalent or enhanced therapeutic or prophylactic effect. Generally. peptidomimetics are structurally similar to the peptide of interest, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983;Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and the like.

As used herein, the term “imaging agent” refers to any agent which may be used in connection with methods for imaging, including but not limited to, imaging an internal region of a subject and/or diagnosing or detecting the presence or absence of a disease or lesion in a cell, tissue, or subject and/or detection of an energy source. Exemplary imaging agents include agents for use in connection with ultrasound, magnetic resonance imaging, radionuclide imaging, or x-ray, including computed tomography, imaging of a subject or sample.

The imaging agent should be coupled to the peptide so as to not interfere with the binding affinity or specificity of the detecting agent for the cancer. The imaging agent may be appended either to the N-terminus, the C-terminus, or an amino acid residue within the sequence.

Attachment of the imaging agent to the peptide may also be achieved by covalent attachment, using any of a variety of methods known to those in the art. For example, the peptide and imaging agent may be linked using bifunctional reagents that are capable of reacting with both the peptide and the imaging agent, thereby forming a bridge between the two.

Attachment may also be achieved by forming a covalent bond directly between a peptide and imaging agent. Any of a variety of standard methods may be used to form a covalent linkage (e.g., formation of a disulfide bond between thiol groups).

The imaging agent may be attached to the peptide with a linker. Linkers include, but are not limited to, amide, urea, acetal, ketal, double ester, carbonyl, carbamate, thiourea, sulfone, thioester, ester, ether, disulfide, lactone, imine, phosphoryl, or phosphodiester linkages; linear, branched, or cyclic amino acid chains of a single amino acid or different amino acids; sugar acids, diamines, dialcohols, and the like. Alternatively, conjugation chemistry can link the imaging agent directly to the peptide (e.g., linking an isothiocyanate group bearing the imaging agent to a free amino group).

The imaging agent may include a radionuclide (a nuclear imaging agent), a radiopaque agent, a radiolucent agent, a contrast agent, a metal, quantum dot, an enzyme, a chelator, or a combination thereof.

In some embodiments, the imaging agent comprises a radionuclide. The radionuclide may be a gamma or positron emitting nuclide (e.g., ¹²³I, ^{99m}Tc and ¹⁸F, ¹²⁴I, respectively). Exemplary radionuclides include: ¹⁸F, ⁴⁷Sc, ⁵¹Cr, ⁶¹Cu, ⁶²Cu, ⁶⁴Cu, ⁶⁷Cu, ⁶⁶Ga, ⁶⁷Ga, ⁶⁸Ga, ⁷⁷Br, ⁷⁶Br, ³⁹Zr, ⁹⁰Y, ⁸⁸Y, ⁹⁷Ru, ^{99m}Tc, ¹⁰³Ru, ¹⁰⁵Rh, ¹⁰⁹Pd, ¹¹¹In, ^{117m}Sn, ¹²⁴I, ¹²⁵I, ¹³¹I, ¹²³I, ¹⁴⁰La, ¹⁴¹Ce, ¹⁵³Sm, ¹⁵³Gd, ¹⁵⁵Gd, ¹⁵⁷Gd, ¹⁴⁹Th, ¹⁵²Th, ¹⁵⁵Th, ¹⁶¹Th, ¹⁶⁵Dy, ¹⁶⁶Dy, ¹⁶⁷Tm, ¹⁶⁸Yb, ¹⁷⁵Yb, ¹⁷⁷Lu, ¹⁷⁹Pm, ¹⁸⁶Re, ¹³³Re, ¹⁹⁸Au, ¹⁹⁹Au, ²⁰³pp, ²¹¹Bi, ²¹²Bi, ²¹³Bi, ²¹⁴Bi, ²²⁵Ac, ²³⁰U, and ²²³Ra. In some embodiments, the radionuclide is a ¹²³I, ^{99m}Tc, ¹⁸F or ¹²⁴I. In select embodiments, the radionuclide is ¹⁸F.

In some embodiments, the imaging agent comprises a contrast agent. MRI contrast agents include supraparamagnetic iron oxide particles, nitroxides, and paramagnetic metal chelates.

In some embodiments, the imaging agent comprises a chelator. Exemplary chelators and chelating groups are known in the art, as described in: WO 98/18496, WO 86/06605, WO 91/03200, WO 95/28179, WO 96/23526, WO 97/36619, PCT/US98/01473, PCT/US98/20182, and U.S. Pat. Nos. 4,899,755, 5,474,756, 5,846,519, and 6,143,274, all of which are hereby incorporated by reference.

Exemplary chelators include: 1,4,7-triazacyclononane-1,4,7-triacetic acid (NOTA); diethylenetriamine pentaacetate (DTPA); 1,4,7,10-tetraazacyclododecane-1,4,7,10-tetrayl)tetraacetic acid (DOTA or tetraxetan); 1,4,7, 10-tetraazacyclododecane-1,4,7-triacetic acid (DO3A); ethylenediaminetetraacetic acid (EDTA); N1,N1′-(Ethane-1,2-diyl)di(ethane-1,2-diamine) (TETA); N,N′-ethylenebis [2-(2-hydroxyphenyl)-glycine] (EHPG); N,N-bis(2-hydroxybenzyl)ethylenediamine-N,N-diacetic acid (HBED); 2,2′,2″-(1,4,7-triazacyclononane-1,4,7-triyl)triacetic acid (NOTA); 1,4,7,10-tetraazacyclotetradecane-1,4,7,10-tetra(methyl tetraacetic acid) (DOTMA); 1,4,7-triazacyclononane macrocycles substituted with phosphonic (NOTP) and phosphinic (TRAP) groups at the amine; bis(2-hydroxybenzyl)ethylenediaminediacetic acid (HBED), a tris(hydroxypyridinone) containing three 1,6-dimethyl-3-hydroxypyridin-4-one groups (THP); the hexadentate tris(hydroxamate) siderophore desferrioxamine-B (DFO); and derivatives thereof, including 5-C1-EHPG, 5-Br-EHPG, 5-Me-EHPG, 5-t-Bu-EHPG, 5-sec-Bu-EHPG, benzo-NOTA 1,4,7-tris (carboxymethyl)-10-(2-hydroxypropyl)-1,4,7,10-tetraazacyclododecane (HP-DO3A); and many others. In some embodiments, the chelator is selected from the group consisting of NOTA, DTPA, DOTA, and DO3A. In select embodiments, the chelator is NOTA.

The chelator may be configured to bind to a radionuclide, a contrast agent, or a metal. Thus, in some embodiments, the imaging agent may comprise a chelator bound to a radionuclide, a contrast agent, or a metal.

Accordingly, further disclosed herein is a conjugate comprising a detecting agent comprising: a peptide derived from thymosin beta-10 (Tβ-10), as described above herein, covalently attached to an imaging agent, wherein the imaging agent is a chelator; and a radionuclide, contrast agent or metal bound to the chelator. In certain embodiments, the conjugate comprises a detecting agent as disclosed herein, wherein the imaging agent is a chelator, and a radionuclide bound to the chelator.

### 3. Compositions

Disclosed herein are compositions comprising the cancer detecting agents described above. The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, com starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration to a subject, and thus administered, including for example, oral, nasal, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

The composition may comprise radiation stabilizers to prevent radiolytic damage to the compound prior to injection. Radiation stabilizers are known in the art, and can include, for example, para-aminobenzoic acid, ascorbic acid, gentisic acid and the like.

The disclosed agents may be in a liposome such that the agent is added to amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

### 4. Methods

The disclosed detecting agents may be used in various methods for detecting, diagnosing, or imaging cancer.

In some embodiments, the cancer comprises a solid tumor. In some embodiments, the cancer is metastatic cancer. The disclosed detecting agent and methods may be used to detect, diagnose, or image a variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In some embodiments, the cancer may comprise breast cancer.

The methods may comprise contacting one or more cells, organs, or tissues with an effective amount of the detecting agent, composition, or conjugate as disclosed herein; and identifying the detecting agent.

The terms “effective amount” as used herein, refer to a sufficient amount of the detecting agents or compositions disclosed herein being administered which will be sufficient to provide satisfactory imaging. For example, when using an aqueous solution of a radionuclide, the dosage is about 1.0 to 100 millicuries. A detectably effective amount may be administered in more than one injection.

The detectably effective amount can vary according to factors such as the degree of susceptibility of the individual, the age, sex, and weight of the individual, idiosyncratic responses of the individual, and the dosimetry. Detectably effective amounts can also vary according to instrument and film-related factors. The amount of detecting agent and, by extension, the imaging agent used for diagnostic purposes and the duration of the imaging study will depend upon the radionuclide, the body mass of the patient, and on the idiosyncratic responses of the patient. Ultimately, the actual dose administered to a patient for imaging purposes, however, is determined by the physician administering treatment.

The detecting agent should be administered so as to remain in the patient for about 1 hour to 10 days, although both longer and shorter time periods are acceptable. Therefore, convenient ampules containing 1 to 10 mL of aqueous solution may be prepared.

The amount of the detecting agent of the present invention which will be effective in the diagnosis, monitoring, or imaging can be determined by standard clinical techniques. In addition, in vivo and/or in vitro assays may optionally be employed to help identify optimal usage ranges.

Detecting agents, compositions, or conjugates disclosed herein can be evaluated for efficacy and toxicity using known methods. For example, the toxicology of a particular agent, composition, or conjugate may be established by determining in vitro toxicity towards a cell line, such as a mammalian, and preferably human, cell line. The results of such studies are often predictive of toxicity in animals, such as mammals, or more specifically, humans. Alternatively, the toxicity of particular peptides in an animal model, such as mice, rats, rabbits, dogs, or monkeys, may be determined using known methods. The efficacy of a particular detecting agent, composition, or conjugate may be established using several recognized methods, such as in vitro methods or animal models. When selecting a model to determine efficacy, the skilled artisan can be guided by the state of the art to choose an appropriate model and administration regime.

In some embodiments, the contacting comprises administering to a subject. The subject may have or be suspected of having cancer. In some embodiments, the subject is human.

Detecting agents, compositions, or conjugates of the present disclosure may be administered to a subject by a variety of methods. In any of the uses or methods described herein, administration may be by various routes known to those skilled in the art, including without limitation oral, inhalation, intravenous, intramuscular, subcutaneous, systemic, and/or intraperitoneal administration to a subject in need thereof. In some embodiments, the peptides or compositions as disclosed herein may be administered by parenteral administration (including, but not limited to, subcutaneous, intramuscular, intravenous, intraperitoneal, intracardiac and intraarticular injections).

In some embodiments, the identifying comprises exposing the subject or a region of the subject to an image scanner; obtaining an image of the subject or the region of the subject; and recognizing the detecting agent. Thus, the methods may comprise a method of imaging cancer in a subject having or suspected of having cancer. Accordingly, in one embodiment, the present disclosure provides methods of detecting the presence of cancer in the subject, thereby diagnosing the subject as having cancer.

Imaging may be carried out in the normal manner, for example by administering a sufficient amount of the detecting agent, or conjugate or composition thereof to provide adequate imaging and then scanning with a suitable imaging or scanning machine, such as a tomograph or gamma camera.

Methods for imaging include but are not limited to magnetic resonance imaging (MRI), computed axial tomography (CAT) scanning, positron emission tomography (PET). ultrasonic imaging, x-rays, radionuclide imaging, single photon emission computed tomography (SPECT), and multiphoton microscopy. Choice of imaging methods are dependent on the use of imaging agent used. For example, for positron emission tomography, positron or gamma-photon emitting radionuclide-based imaging agents or chelators thereof, may be used.

The methods may further comprise quantifying or measuring lesion load in the subject. Quantitation may be achieved using standard methods such as drawing regions of interest around patient organs or tissues positive in the image for the detecting agent and thereby determining the amount of radioactivity within that region. This method of quantifying the distribution of radiolabeled tracer molecules in patients is particularly accurate when PET images are used. The methods of the present invention may also further comprise comparing the image to an image of a control subject not having cancer. The method may be used to monitor the progression of a cancer by comparing a recently obtained image to an image obtained earlier in time to determine whether there is an increase or decrease in the amount of cancer lesions or the size of a lesion. Alternatively, the image may be obtained after treatment or therapy, and the image may be compared to an image obtained prior to treatment or therapy to follow effectiveness of the treatment or therapy. Accordingly, the image could be used to monitor the response of the subject to treatment or therapy.

The detecting agents of the present disclosure may also be used to identify and quantitate cancer by in vitro methods. The detecting agents of the present disclosure may be used to identify cancer in a tissue sample obtained from a subject for diagnosis or treatment of the subject and for monitoring the progression of a subject's condition or the response of the subject to therapy

### 5. Kits

In another aspect, the disclosure provides kits comprising a detecting agent as disclosed herein. In some embodiments, the imaging agent is a chelator and a radionuclide. Descriptions of the detecting agents, including chelator and radionuclides provided elsewhere herein are suitable for use with the disclosed kits.

The kits may further include administration reagents or devices, negative and positive control samples, additional therapeutic agents, containers (e.g., microcentrifuge tubes), detection and analysis instruments, software, instructions, and the like.

The kits can comprise instructions for using the components of the kit. The instructions are relevant materials or methodologies pertaining to the kit. The materials may include any combination of the following: background information, list of components, brief, or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks, syringes).

The kits provided herein are in suitable packaging. Suitable packaging includes, but is not limited to, vials, bottles, jars, flexible packaging, and the like. Individual member components of the kits may be physically packaged together or separately.

### 6. Examples

### Example 1

### Preparation of Tβ10-Based Peptides for PET Imaging

Peptides were prepared using standard solid phase peptide synthesis strategies using side-chain protected amino-acid anchored resin and amino acids. Site-specific addition of the chelator NOTA was carried out via protecting group methodologies. Products were purified using reverse-phase high-performance liquid chromatography (RP-HPLC) and further characterized by electrospray ionization mass spectrometry (ESI-MS).

Two peptides comprising the same sequence (SEQ ID NO: 1) but with different sites of imagining agent (NOTA) were targeted for initial study, as shown below.

Pep61:
(SEQ ID NO: 2)
A-D-K(NOTA)-P-D-M-G-E-I-A-S-F-D-K
Pep62:
(SEQ ID NO: 3)
A-D-K-P-D-M-G-E-I-A-S-F-D-K(NOTA)

### Example 2

### Biological Assessment Pep61 and Pe-62

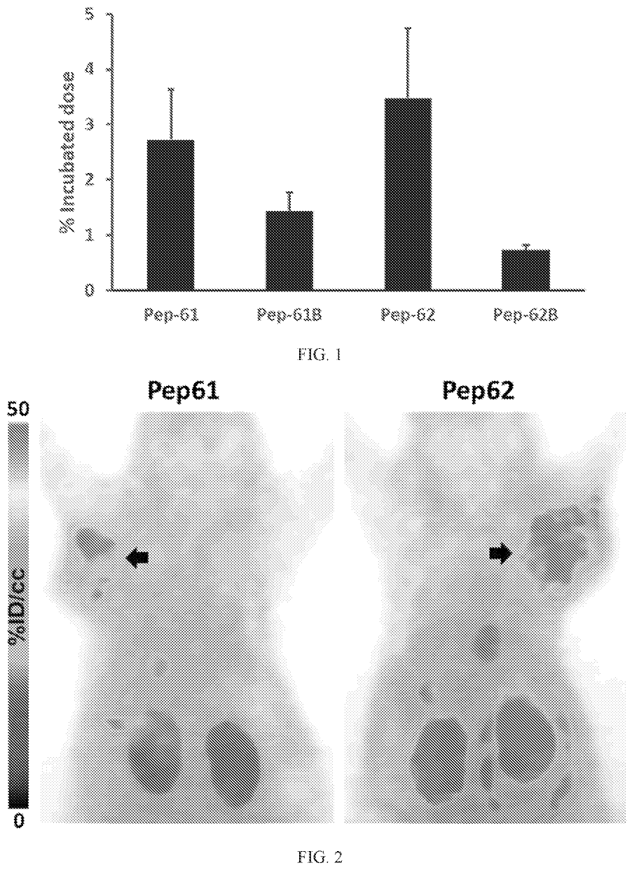

Prepared peptides were first analyzed in vitro using the human triple negative breast cancer cell line, MDA-MB-231. In brief, cells were detached using non-enzymatic buffer and 0.5 million cells per tube will be incubated with 74 KBq (2 μCi) of ¹⁸F-labeled peptide in 100 μL phosphate-buffered saline (PBS) at 37° C. Experiments were done in triplicate and repeated three times to ensure reproducibility. Blocking was carried out using a 10-fold molar equivalence of the corresponding non-radioactive peptide analogue. The integrity of the radiolabeled compounds and their extent of decomposition was analyzed by RP-HPLC. As shown in FIG. 1, both Pep61 and Pep62 taken up by MDA-MB-231 cells in a specific manner.

All animal studies were carried out per regulations set forth and approved by the Johns Hopkins Animal Care and Use Committee at the Johns Hopkins Molecular Imaging Service Center. Female, 6-8 weeks old, immunodeficient (NOG) mice were inoculated with MDA-MB-231 (2 million) cells in 100 μL of Hank's balanced salt solution in the top mammary fat pad. Tumors averaging 200-300 mm³ (typically 14 days after inoculation) were used for in vivo imaging and ex vivo biodistribution studies.

For PET imaging, mice were intravenously injected with 9-10 MBq (240-270 μCi) of the radiolabeled peptide in 200 μL of saline. Whole body PET imaging (2 beds; 15 min per bed) was carried out using a SEDCAL SuperArgus small animal PET/CT scanner 30 min post tracer injection. Following PET image acquisition, a CT scan (512 projections) was obtained on the same system for anatomical co-registration. PET data were reconstructed using the 3-dimensional ordered-subsets expectation maximization (3D-OSEM) algorithm and corrected for dead time and radioactive decay. Final data visualization and image generation was carried out using the Amira (FEI) software. Quantitative in vivo image analysis was done in AMIDE 1.0.5 (SourceForge) using ellipsoid region-of-interest volume integrations. Percentage of injected dose per cubic centimeter of tissue (% ID/cc) was calculated based on an external calibration factor obtained from a known quantity of radioactivity.

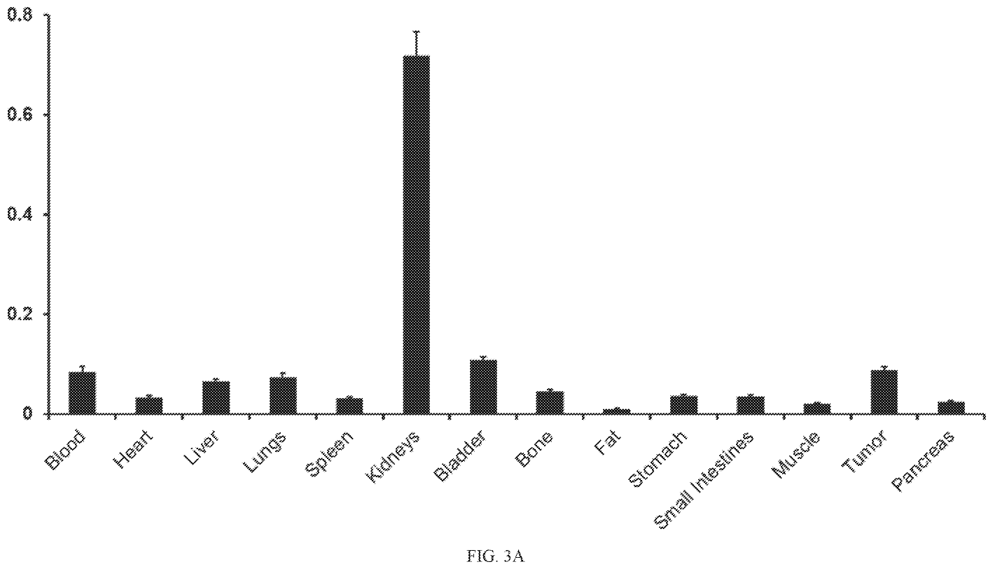

As shown in FIG. 2, Pep61 and Pep62 demonstrated clear accumulation in breast tumors, demonstrating the applications of these compounds for targeted breast cancer imaging. Pep62showed higher and more uniform uptake compared to Pep61, possibly indicating higher tolerance for modifications to the C-terminus of the peptide.

For biodistribution studies, mice (n=5 per peptide) were injected with 1.5-1.8 MBq (40-50 μCi) of tracer in 100 μL of saline intravenously. Ex vivo biodistribution studies were performed at the same time points as the imaging studies (30 min after administration of the tracer) for comparative quantitative analysis. Blood, liver, spleen, heart, lungs, kidneys, small intestines, large intestines, stomach, muscle, fat, bone, bladder, and tumors were retrieved, weighed, and counted on an automated gamma counter (1282 Compugamma CS). The percentage of injected dose per gram of tissue (% ID/g) was calculated, corrected for signal-decay, and normalized to external standards. Statistical analysis of obtained in vivo images as well as in vitro and ex vivo biodistribution data was carried out in GraphPad Prism 6.0 and Microsoft Excel 2016. An unpaired 2-tailed t test will be used in each case to determine statistical significance set when p<0.05.

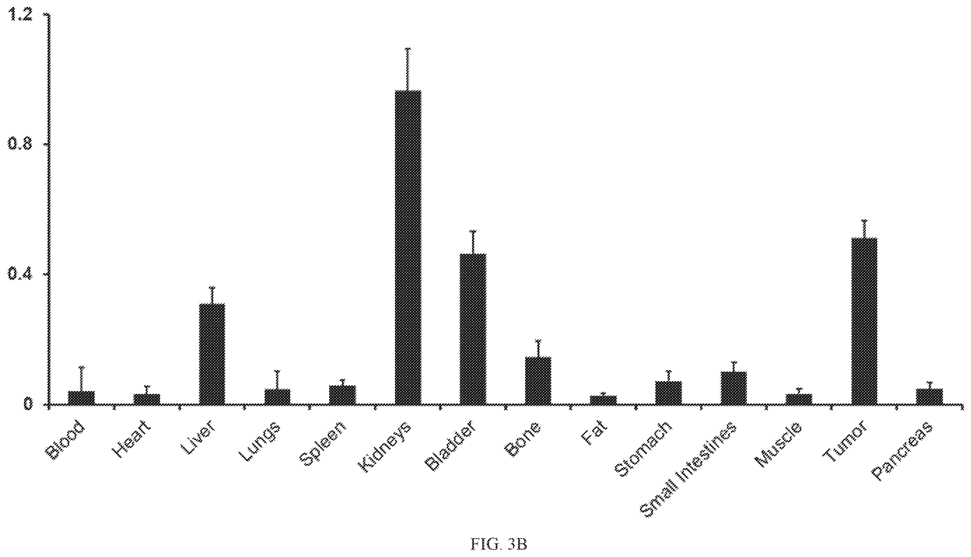

As shown in FIGS. 3A and 3B, Pep61 and Pep62 showed uptake in MDA-MB-231 tumors as well as non-specific retention in clearance organs. Pep62 showed higher overall tumor uptake, as well as tumor-to-blood and tumor-to-muscle ratios compared to Pep61. Thus, the peptides specifically targeted the breast tumors, irrespective of the presence of hormone receptors (ER, PR, and HER2).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. An agent for detecting cancer, comprising a peptide derived from thymosin beta-10 (Tβ-10) covalently attached to an imaging agent,
wherein the peptide comprises less than twenty amino acids.

2. The agent of claim 1, wherein the peptide comprises the amino acid sequence
(SEQ ID NO: 1)
ADKPDMGEIASFDK.

3. The agent of claim 1 or claim 2, wherein the imaging agent is selected from the group consisting of a radionuclide, a chelator, a radiopaque agent, a radiolucent agent, a contrast agent, a metal, quantum dot, or a combination thereof.

4. The agent of any of claims 1-3, wherein the imaging agent comprises a chelator.

5. The agent of claim 3 or claim 4, wherein the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

6. The agent of any of claims 1-5, wherein the imaging agent comprises a radionuclide.

7. The agent of any of claims 1-6, wherein the radionuclide is a gamma or positron emitting radionuclide.

8. The agent of any of claims 3-7, wherein the radionuclide is ¹²³I, ^{99m}Tc, ¹⁸F or ¹²⁴I.

9. The agent of any of claims 3-8, wherein the radionuclide is ¹⁸F.

10. A composition comprising the agent of any of claims 1-9.

11. A conjugate comprising:
an agent of claim 1, wherein the imaging agent is a chelator; and
a radionuclide bound to the chelator.

12. The conjugate of claim 11, wherein the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

13. The conjugate of claim 11 or claim 12, wherein the radionuclide is a gamma or positron emitting radionuclide.

14. The conjugate of any of claims 11-13, wherein the radionuclide is ¹⁸F.

15. A method of detecting or diagnosing cancer comprising:
contacting one or more cells, organs, or tissues with an effective amount of the agent of any of claims 1-9, a composition of claim 10, or a conjugate of any of claims 11-14; and
identifying the agent.

16. The method of claim 15, wherein the contacting comprises administering to a subject.

17. The method of claim 16, wherein the identifying comprises:
exposing the subject or a region of the subject to an image scanner;
obtaining an image of the subject or the region of the subject; and
recognizing the agent.

18. The method of claims 16 or claim 17, wherein the subject has or is suspected of having cancer.

19. The method of any of claims 15-18, wherein the cancer comprises a solid tumor.

20. The method of any of claims 15-19, wherein the cancer comprises breast cancer.

21. A kit comprising:
an agent of claim 1, wherein the imaging agent is a chelator, and
a radionuclide.

22. The kit of claim 21, wherein the chelator is selected from the group consisting of 1,4,7-triazacyclononane-1,4,7-triacetic acid (NOTA), diethylenetriamine pentaacetate (DTPA), 1,4,7,10-tetraazacyclododecane-1,4,7,10-tetrayl)tetraacetic acid (DOTA), and 1,4,7,10-tetraazacyclododecane-1,4,7-triacetic acid (DO3A).

23. The kit of claim 21 or 22, wherein the radionuclide is a gamma or positron emitting radionuclide.

24. The kit of any of claims 21-23, wherein the radionuclide is ¹⁸F.


SEQUENCE LISTING (3 sequences)

SEQ ID NO: 1 - protein, 14 aa, synthetic construct
ADKPDMGEIASFDK

SEQ ID NO: 2 - protein, 14 aa, synthetic construct
  SITE 3: note = 1,4,7-triazacyclononane-1,4,7-triacetic acid labeled lysine
ADKPDMGEIASFDK

SEQ ID NO: 3 - protein, 14 aa, synthetic construct
  SITE 14: note = 1,4,7-triazacyclononane-1,4,7-triacetic acid labeled lysine
ADKPDMGEIASFDK